(19) United States
(12) Patent Application Publication
(10) Pub. No.: US 2026/0273170 A1
(43) Pub. Date: Sep. 17, 2026

(54) A CANNULA ASSEMBLY OF A MEDICAMENT DELIVERY SYSTEM

(71) Applicant: SHL Medical AG, Zug (CH)

(72) Inventors: René Zander, Zug (CH); Lukas Bannwart, Zug (CH); Samuel Wyler, Zug (CH)

(21) Appl. No.: 19/168,369

(22) PCT Filed: Mar. 22, 2024
(86) PCT No.: PCT/EP2024/057725

§ 371 Date: Sep. 24, 2025

Related U.S. Application Data
(60) Provisional application No. 63/455,093, filed on Mar. 28, 2023.
(60) Provisional application No. 63/455,099, filed on Mar. 28, 2023.
(60) Provisional application No. 63/455,103, filed on Mar. 28, 2023.

(30) Foreign Application Priority Data
May 15, 2023 (EP) 23173392.4

(51) Int. Cl. A61M 5/14

(52) U.S. Cl. CPC A61M 5/1413

(57) ABSTRACT

The present disclosure provides a cannula assembly of a medicament delivery system, the cannula assembly for being fluidly connected between an infusion container and an infusion tube. The cannula assembly includes a housing extending between a container end configured to be operably attached to the infusion container and a tube end configured to be operably attached to the infusion tube along a longitudinal axis. The cannula assembly further includes a cannula hub movably arranged at least partially within the housing. The cannula assembly further includes a cannula comprising a cannula body having a tube section configured to be operably attached to an infusion tube and a container section configured to be operably attached to an infusion container. The container section of the cannula body is attached to the cannula hub The cannula hub is movable relative to the housing in the direction of the longitudinal axis between an initial position where the container section of the cannula body of the cannula is fully received in the housing and a connected position where the cannula body of the cannula is positioned at least partially outside of the container end of the housing.

### TECHNICAL FIELD

The present disclosure generally relates to a cannula assembly for being fluidly connected between an infusion container and an infusion tube, and particularly a cannula assembly with a housing and a cannula hub movable relative to the housing.

### BACKGROUND

Usually in the administration of the high volume and/or viscosity medicament are carried out in the medical professional facility, e.g., a hospital; and patients are assisted by medical professionals. For example, the administration of the high-volume medicament can be done by an intravenous infusion. In this case, the medical professional will bring an infusion bag to the patient, hang the infusion bag, connect a cannula between the infusion bag and an infusion tube to create a fluid connection between the medicament and the infusion tube, place the infusion needle to the patient's body, and connect the infusion tube to the infusion needle.

Nowadays many medicament delivery devices such as auto-injectors or infusion pumps can be designed for the self-administration of a medicament by patients without formal medical training so that patients do not need to visit the medical professional facility often. Thus, multiple automatic functions are desirable to avoid any potential handling error.

For administrating high volume and/or viscosity medicament, or multiple different medicaments, the users might need to take more steps to perform the self-administration of a medicament. For example, it is common for high volume and/or viscosity medicament to be stored away from the medicament delivery member to avoid any potential contamination and leakage. Thus, there is another step of creating the fluid connection between the medicament and the medicament delivery member.

Thus, it is desirable to simplify the steps of the user, e.g., both the patients and caregivers, for example, to simplify the steps of establishing a fluid connection between a medicament container and a medicament delivery member.

### SUMMARY

The invention is defined by the appended claims, to which reference should now be made.

There is hence provided a cannula assembly of a medicament delivery system, the cannula assembly for being fluidly connected between an infusion container and an infusion tube; the cannula assembly comprising:
a housing extending between a container end configured to be operably attached to the infusion container and a tube end configured to be operably attached to the infusion tube along a longitudinal axis;
a cannula hub movably arranged at least partially within the housing;
a cannula comprising a cannula body having a tube section configured to be operably attached to an infusion tube and a container section configured to be operably attached to an infusion container;
wherein the container section of the cannula body is attached to the cannula hub; and
wherein the cannula hub is movable relative to the housing in the direction of the longitudinal axis between an initial position where the container section of the cannula body of the cannula is fully received in the housing and a connected position where the cannula body of the cannula is positioned at least partially outside of the container end of the housing.

The cannula assembly is suitable for being used in the medicament delivery system that is configured to deliver high-volume, and/or high-viscosity, and/or multiple medicaments. The cannula assembly is configured to create the fluid connection between the infusion container and the infusion tube only when the patient plans to receive the medicament, e.g., a movement of the cannula hub needs to be triggered.

Preferably, according to one embodiment, the container section of the cannula body comprises a sharp end for opening a seal of the infusion container. The tube section, on the other hand, doesn't need to be sharp as the tube section is only configured to be connected to the infusion tube, not piercing patient's skin or a seal.

Preferably, according to another embodiment, the cannula hub comprises a fluid channel being coaxial with the cannula body; and the tube section of the cannula body is fully received within the cannula hub.

Preferably, according to another embodiment, the cannula hub comprises a protruding section protruding from the housing such that the cannula hub is configured to be moved from the initial position to the connected position manually by a user by moving the protruding section relative to the housing.

As there is no component needed to cause the movement of the cannula hub, the cost of the cannula assembly can be reduced.

Preferably, according to another embodiment, the protruding section of the cannula hub is positioned through a longitudinally extending slot in a wall of the housing.

Alternatively, according to another embodiment, the cannula assembly comprises:
a biasing member extending in the direction of the longitudinal axis between a surface of the housing and a surface of the cannula hub such that the cannula hub is biased towards the connected position by the biasing member; and
a cannula hub lock comprising a surface facing away from the container end of the housing;
   wherein the cannula hub comprises a counter surface directed towards the container end of the housing; wherein the cannula hub lock is immovable relative to the housing in the direction of the longitudinal axis;
   wherein the surface of the cannula hub lock is engaged with the counter surface of the cannula hub in a locked position such that the cannula hub is prevented from being biased into the connected position and is disengaged from the counter surface of the cannula hub in a released position such that the cannula hub is biased into the connected position by the biasing member.

In this embodiment, the movement of the cannula hub is caused by the biasing member such that the cannula can be more precisely connected to the infusion container.

Preferably, according to another embodiment, the cannula assembly comprises a lock holder having a body movable relative to the housing between a first position and a second position.

Preferably, according to another embodiment, the cannula hub lock is operably connected to the lock holder when the lock holder is in the first position and is not connected to the lock holder when the lock holder is in the second position.

Preferably, according to another embodiment, the body of the lock holder comprises an inner section positioned within the housing and an outer section positioned outside of the housing.

Preferably, according to another embodiment, the body of the lock holder is movable relative to the housing between the first position and the second position in the direction transverse to the longitudinal axis.

Alternatively, according to another embodiment, the body of the lock holder is movable relative to the housing between the first position and the second position in the direction of the longitudinal axis.

Preferably, according to another embodiment, the biasing member is a spring.

Alternatively, according to another embodiment, the biasing member is a resilient arm.

Preferably, according to another embodiment, the surface of the cannula hub lock is movable from the locked position to the released position in the direction transverse to the longitudinal axis.

Preferably, according to another embodiment, the surface of the cannula hub lock is radially flexible from the locked position to the released position relative to the longitudinal axis.

Preferably, according to another embodiment, the cannula hub lock comprises a flexible arm extending from the housing; wherein the flexible arm comprises a ledge having the surface of the cannula hub lock.

Preferably, according to another embodiment, the lock holder is an outer sleeve movable relative to the housing in the direction of the longitudinal axis between a first position where the outer sleeve is circumferentially adjacent to the flexible arm and a second position where the outer sleeve is circumferentially offset from the flexible arm.

Preferably, according to another embodiment, the outer section of the lock holder extending in the direction of the container end of the housing and beyond the container end of the housing in the direction of the longitudinal axis such that the outer sleeve is moved by the infusion container from the first position to the second position when the container end of the housing is attached to the infusion container.

Alternatively, according to another embodiment, the cannula hub lock comprises a clip having a clip body enclosing the cannula hub.

Preferably, according to another embodiment, the clip body comprises a ledge having the surface of the cannula hub lock.

Preferably, according to another embodiment, the clip body is circumferentially expandable relative to the longitudinal axis such that the surface of the cannula hub lock is moved from the lock position to the released position when the clip body is circumferentially expanded outwards from the longitudinal axis.

Preferably, according to another embodiment, the lock holder is movable relative to the clip from a first position where the inner section of the body of the lock holder is coupled to the clip such that the clip body is prevented from expanding to a second position where the inner section of the body of the lock holder is decoupled from the clip such that the clip body is allowed to expand.

Preferably, according to another embodiment, the inner section of the body is engaged with the clip body when the inner section of the body of the lock holder is coupled to the clip; and wherein the inner section of the body of the lock holder is disengaged from the clip body when the inner section of the body is decoupled from the clip.

Preferably, according to another embodiment, the inner section of the body is engaged with the clip body when the inner section of the body of the lock holder is coupled to the clip; and wherein the inner section of the body of the lock holder is disengaged from the clip body when the inner section of the body is decoupled from the clip.

Alternatively, according to another embodiment, the cannula hub lock comprises two extensions extending in the direction transverse to the longitudinal axis; wherein the two extensions are configured to be spaced apart from one another; wherein the clip body is extending in a circumferential direction around the longitudinal axis from one of the two extensions to the other of the two extensions; wherein the inner section of the body is engaged with the two extensions when the inner section of the body is coupled to the clip; and wherein the inner section of the body is disengaged from the extensions when the inner section of the body is decoupled from the clip.

Alternatively, according to another embodiment, the lock holder is an integral part of the cannula hub lock.

Preferably, according to another embodiment, the outer section of the body of the lock holder is a user-accessible button such that the lock holder is moved by a user of the cannula assembly manually to cause the cannula hub lock to be moved from the locked position to the released position.

Preferably, according to another embodiment, the cannula hub lock is configured to be manually moved by the user from the lock position to the released position.

Alternatively, according to another embodiment, the outer section of the body of the lock holder is operably connected to a power source of the medicament delivery device.

Preferably, according to another embodiment, the cannula hub lock is configured to be moved by the power source of the medicament delivery device from the lock position to the released position.

Preferably, according to another embodiment, the outer section of the body of the lock holder is configured to be positioned within a gas-tight chamber such that the lock holder is moved by a change of the pneumatic pressure within the gas-tight chamber to cause the cannula hub lock to be moved from the locked position to the released position.

Preferably, according to another embodiment, the housing comprises a container fastener configured to be operably attached to the infusion container.

Preferably, according to another embodiment, the container fastener is nearby the container end of the housing.

Preferably, according to another embodiment, the container fastener is at least one of a thread, a bayonet connector, and a snap-fit connector.

Preferably, according to another embodiment, the container section of the cannula is sealed by a sheath.

Alternatively, or additionally, according to another embodiment, the container end of the housing is sealed by a membrane.

Preferably, according to another embodiment, the housing comprises a hub fastener.

Preferably, according to another embodiment, the cannula hub comprises a counter fastener attached to the hub fastener of the housing when the cannula hub is in the connected position.

Preferably, according to another embodiment, the hub fastener of the housing and the counter fastener of the cannula hub are configured to form a snap-fit attachment when the cannula hub is in the connected position.

Preferably, according to another embodiment, the cannula assembly is configured to be used in a medicament delivery system comprising an infusion container, an infusion tube, a medicament delivery member, and the cannular assembly.

Preferably, according to another embodiment, the medicament delivery system comprises a medicament delivery device having a power source to automatically expel the medicament contained within the infusion container; the infusion tube; and the medicament delivery member.

Preferably, the power source of the medicament delivery device can be a motor-driven gear set, a peristaltic pump, a magnet-based pressing pump, a spring, or a fluid pressure power source.

Preferably, according to another embodiment, the medicament delivery device comprises a reusable body comprising the fluid pressure power source; a container carrier; and an infusion container having a body with a fluid outlet. The body comprises a flexible portion. The infusion container is at least partially arranged within the container carrier. The container carrier comprises a fluid-tight chamber. The fluid-tight chamber comprises an inlet. The inlet is configured to fluidly connect to an outlet of the fluid pressure power source of the reusable body of the medicament delivery device. The fluid-tight chamber is coupled to the flexible portion of the body of the infusion container so that when the inlet receives output fluid from the fluid pressure power source, the output fluid flows into the fluid-tight chamber and the medicament contained within the infusion container is pressed out under the pressure of the output fluid. The fluid outlet is configured to be connected to the cannula assembly as disclosed above, such that the medicament can be delivered to a medicament delivery site through the fluid outlet, the cannula assembly, the infusion tube, and a medicament delivery member of the medicament delivery device.

Preferably, according to another embodiment, the medicament delivery device comprises a cassette removably attached to the reusable body.

Preferably, according to another embodiment, the cassette comprises the container carrier.

Preferably, according to another embodiment, the cassette comprises a cassette housing.

Preferably, according to another embodiment, the cassette housing is configured to be attached to the reusable body of the medicament delivery device.

Preferably, according to another embodiment, the container carrier is arranged within the cassette housing.

Preferably, according to another embodiment, the body of the infusion container comprises a flexible bag.

Preferably, according to another embodiment, the fluid pressure power source is a pneumatic power source or a hydraulic power source. The pneumatic power source is configured to output gas to the infusion container and expel the medicament contained within the infusion container by increasing the gas pressure around the fluid-tight chamber. The hydraulic power source is configured to output liquid to the infusion container and expel the medicament contained within the infusion container by increasing the liquid pressure around the fluid-tight chamber. When the fluid pressure power source is the pneumatic power source, the fluid-tight chamber is gas-tight. When the fluid pressure power source is a hydraulic power source, the fluid-tight chamber can be either gas-tight or liquid-tight.

Therefore, a medicament delivery device comprising the infusion container as disclosed in this disclosure can provide a drive system for a pneumatic pressure-driven or hydraulic pressure-driven medicament delivery device capable of delivering one or more medicaments in a desired sequence, each at a desired flow rate of delivery of the medicament. The system does not require prior knowledge of the volume and/or viscosity of each medicament. Therefore, the reusable body of the medicament delivery device can be used for different medicaments contained within different disposable cassettes. The infusion container can be filled with medication by a pharmacy (“fill at point of use”) or by a drug maker (“prefilled”). “, e.g., in a hospital or infusion center compounding suite) or by a drug maker (“prefilled”). During use, air can be added to the interior of the cassette at a known mass flow rate to collapse the flexible container inside the cassette and deliver its contents to a patient at a known and controllable volumetric flow rate.

Preferably, according to another embodiment, the fluid-tight chamber is a secondary flexible bag that fully encloses the infusion container. This could be accomplished via a multiple-layer bag assembly with one port as the fluid outlet and the other port as the inlet of the fluid-tight chamber.

Preferably, according to another embodiment, the infusion tube comprises a tube valve.

Preferably, according to another embodiment, the tube valve is an in-line valve included in the infusion tube.

Preferably, according to another embodiment, the tube valve of the infusion tube is a one-way valve so that the fluid cannot flow towards the infusion container through the infusion tube.

Preferably, according to another embodiment, the tube valve of the infusion tube is an umbrella valve or a Belleville valve or a ball valve or a pinch valve.

Preferably, according to another embodiment, the body of the infusion container is at least partially accommodated within the fluid-tight chamber.

Preferably, according to another embodiment, the inlet of the fluid-tight chamber is the outlet of the fluid-tight chamber.

Preferably, according to another embodiment, the container carrier comprises a container chamber configured to at least partially accommodate the infusion container.

Preferably, according to another embodiment, the fluid-tight chamber is inflatable. The fluid-tight chamber is adjacent to the body so that the output fluid from the fluid pressure power source is configured to flow into the fluid-tight chamber and inflate the fluid-tight chamber to press on the infusion container.

Preferably, according to another embodiment, the inlet of the fluid-tight chamber comprises a valve.

Preferably, according to another embodiment, the valve of the inlet of the fluid-tight chamber is a one-way valve so that the fluid can only pass the one-way valve for entering the fluid-tight chamber.

Preferably, according to another embodiment, the medicament delivery device comprises at least two infusion containers.

Preferably, according to another embodiment, the medicament delivery device comprises at least two container carriers.

Preferably, according to another embodiment, the at least two container carriers the at least two infusion containers respectively.

Preferably, according to another embodiment, the cassette comprises the at least two infusion containers.

Preferably, according to another embodiment, the cassette comprises the at least two container carriers.

Preferably, according to another embodiment, the medicament delivery device comprises one container carrier; and the container carrier comprises at least two fluid-tight chambers.

Preferably, according to another embodiment, the at least two fluid-tight chambers container carriers accommodate the at least two infusion containers respectively.

Preferably, according to another embodiment, the cassette comprises one container carrier; and the container carrier comprises at least two fluid-tight chambers.

Preferably, according to another embodiment, each infusion container is provided with a fluidically separate connection to the patient in a manner by which separate medications do not mix during administration unless desired at the patient site.

Preferably, according to another embodiment, a one-way valve is arranged between the at least two fluid-tight chambers.

Preferably, according to another embodiment, the one-way valve arranged between the at least two fluid-tight chambers is a single-use valve, e.g., a frangible valve.

Preferably, according to another embodiment, only one fluid-tight chamber of one of the at least two container carriers comprises the inlet that is configured to be fluidly connected to the fluid pressure power source.

Preferably, according to another embodiment, the one-way valve between the two fluid-tight chambers is configured to open when a first fluid pressure threshold is reached. The valve of the inlet of the fluid-tight chamber that is configured to be fluidly connected to the fluid pressure power source is configured to open when a second fluid pressure threshold is reached. The first fluid pressure threshold is equal to or greater than the second fluid pressure threshold.

Preferably, according to another embodiment, the fluid-tight chamber comprises a release valve configured to release the fluid flowing within the fluid-tight chamber out of the fluid-tight chamber.

Preferably, according to another embodiment, the release valve is configured to release the fluid that flows from the fluid pressure power source and accumulates within the fluid-tight chamber out of the fluid-tight chamber to the ambient around the medicament delivery device or a receiving container included in the medicament delivery device when the fluid pressure reaches a predetermined threshold.

Preferably, according to another embodiment, the release valve is connected to an emergency button. The release valve is configured to release the fluid flowing within the fluid-tight chamber out of the fluid-tight chamber and to the ambient around the medicament delivery device or a receiving container included in the medicament delivery device when the emergency button is activated, thus, to cease the motivation of the medicament contained within the container.

Preferably, according to another embodiment, the emergency button can be pressed or pulled, or slid, or twisted relative to the container carrier or the reusable body of the medicament delivery device for activating the emergency button.

Preferably, according to another embodiment, the valve of the inlet of the fluid-tight chamber that is configured to be fluidly connected to the fluid pressure power source is configured to open when a second fluid pressure threshold is reached. The predetermined threshold is greater than the second fluid pressure threshold.

Preferably, according to another embodiment, the predetermined threshold is greater than the first fluid pressure threshold.

Preferably, according to another embodiment, the fluid-tight chamber of the container carrier is at least partially made of rigid material.

Preferably, according to another embodiment, the fluid-tight chamber comprises a pressure sensor.

Preferably, according to another embodiment, the container carrier comprises a position sensor configured to detect the position of the infusion container.

Preferably, according to another embodiment, the position sensor of the infusion container is configured to monitor the pressure level within the fluid-tight chamber by monitoring the position of the infusion container.

Preferably, according to another embodiment, the medicament delivery device is portable.

Preferably, according to another embodiment, the medicament delivery device is body-worn, underneath or over-clothing.

Preferably, according to another embodiment, the medicament delivery device is an injection device, e.g., an infusion device or an on-body injector.

Preferably, according to another embodiment, the medicament delivery device is configured to perform an intravenous injection, a subcutaneous injection, or an intramuscular injection.

Preferably, according to another embodiment, the medicament delivery member is an injection needle or an insertion needle with a soft cannula.

Preferably, according to another embodiment, the medicament delivery device comprises a reusable body, a changeable cannula assembly and a changeable medicament delivery member.

Preferably, according to another embodiment, the reusable body of the medicament delivery device comprises the fluid pressure power source connected to the inlet of the fluid-tight chamber of the container carrier. The fluid outlet is operably connected to the medicament delivery member.

Preferably, according to another embodiment, the medicament delivery device comprises a processor electrically connected to the fluid pressure power source, an electrical power source connected to the processor. The processor and the electrical power source are accommodated within the reusable body.

Preferably, according to another embodiment, the fluid-tight chamber is adjacent to the fluid-pressure power source. The fluid pressure power source is fluidly connected to the inlet of the fluid-tight chamber directly.

Preferably, according to another embodiment, a transmission tube is arranged between the fluid pressure power source and the inlet of the fluid-tight chamber. The fluid pressure power source is fluidly connected to the inlet of the fluid-tight chamber via the transmission tube.

Preferably, according to another embodiment, the medicament delivery device comprises a user interface attached to the reusable body.

Preferably, according to another embodiment, the user interface is electrically connected to the processor.

Preferably, according to another embodiment, the user interface is a button protruding from an outer surface of the reusable body.

Preferably, according to another embodiment, the user interface is a screen or touch panel arranged on an outer surface of the reusable body.

Preferably, according to another embodiment, the medicament delivery device comprises a wireless communication receiver connected to the processor.

Preferably, according to another embodiment, the medicament delivery device comprises a wireless communication transmitter connected to the processor.

Preferably, according to another embodiment, the wireless communication receiver is configured to receive a wireless signal from a remote device or an information tag.

Preferably, according to another embodiment, the wireless communication transmitter is configured to transmit a wireless signal to a remote device or an information tag.

Preferably, according to another embodiment, the pressure sensor and/or the position sensor of the piston of the fluid-tight measure chamber and/or the position sensor of the container carrier is electrically connected to the processor.

Preferably, according to another embodiment, the processor is configured to control the fluid pressure power source to output fluid into the fluid-tight chamber according to a signal from the pressure sensor and/or the position sensor of the piston of the fluid-tight measure chamber and/or the position sensor of the container carrier.

Furthermore, when the medicament delivery member is an injection needle or an insertion needle with a soft cannula, the system can also detect a needle removal when a pressure drop within the fluid-tight chamber is detected.

Preferably, according to another embodiment, an intentional breakpoint can be arranged close to the terminating needle end. For example, such that if the infusion tube were to be pulled in a fashion that would normally dislodge the needle from the patient skin, the tube would instead break at this joint separating the infusion tube from the needle end.

This break would have the effect of removing the pressure drop associated with the needle and subcutaneous tissue backpressure. Thus, for a medication delivered at a controlled flow rate, the upstream drive pressure would decrease.

With a drive system capable of continuously monitoring pressure and/or flow rate of medication leaving the source container, these sudden flow changes would be detectable and would indicate detachment of the infusion tube from the terminating needle, presenting an error condition that would stop the injection.

Preferably, according to another embodiment, the fluid pressure power source is connected to the fluid-tight chambers of the two container carriers; and wherein the processor is configured to control the fluid pressure power source to selectively output fluid into at least one of the fluid-tight chambers.

Preferably, according to another embodiment, the processor is configured to control the fluid pressure power source to selectively output fluid into at least one of the fluid-tight chambers according to a signal from the pressure sensor and/or the position sensor of the piston of the fluid-tight measure chamber and/or the position sensor of the container carrier.

Preferably, according to another embodiment, the processor is configured to control the fluid pressure power source to output a certain amount of fluid. The certain amount is predetermined or is dependent on a signal from the pressure sensor and/or the position sensor of the piston of the fluid-tight measure chamber and/or the position sensor of the container carrier; thereby only the certain amount of medicament contained within the infusion container can be pressed out of the fluid outlet.

Preferably, according to another embodiment, the fluid pressure power source comprises a piezo pump configured to cause fluid to output from the fluid pressure power source. Furthermore, the piezo pump is preferably used as the pneumatic power source. Using a piezo pump can be advantageous as it can provide quiet or silent operation and can provide lower power operation when compared to motor-driven solutions.

Preferably, according to another embodiment, the fluid pressure power source comprises a motor-based fluid pump configured to cause fluid to output from the fluid pressure power source.

Preferably, according to another embodiment, the fluid pressure power source comprises a pressurized fluid canister configured to cause fluid to output from the fluid pressure power source.

Preferably, according to another embodiment, when the fluid pressure power source is the pneumatic power source, the pneumatic power source comprises a pressurized gas canister.

Preferably, according to another embodiment, when the fluid pressure power source is the pneumatic power source, the pneumatic power source is a well-controlled diaphragm pump when lower cost or complexity is desired.

Preferably, according to another embodiment, when the fluid pressure power source is the pneumatic power source, the pneumatic power source comprises an electronics engine, e.g., a MEMS engine, and a liquid substance. In this example, the engine is configured to trigger an electrochemical reaction of the liquid substance to generate propellant gas.

Preferably, according to another embodiment, the fluid pressure source is a pneumatic power source. In this embodiment, the fluid outputted from the fluid pressure power source is gas, e.g., air or nitrogen.

Preferably, according to another embodiment, the fluid pressure source is a hydraulic power source. In this embodiment, the fluid outputted from the fluid pressure power source is liquid, e.g., water or oil.

Preferably, according to another embodiment, the cannula assembly comprises a penetrable cap membrane tightly connected to a tip of the cannula, forming a cap around the tip of the cannula, before a soft membrane sealing the container end of the housing is pierced.

Preferably, according to another embodiment, the cannula has a blunt tip and a side opening.

Preferably, according to another embodiment, the penetrable cap membrane is a compressible membrane covering the cannula and creating a sterile barrier around the cannula before a soft membrane sealing the container end of the housing is pierced.

Preferably, according to another embodiment, a bag port comprises a cap axially pierced but in which some remaining matter needs to be pierced by the cannula to access the inside of the medicament bag.

Preferably, according to another embodiment, the cannula assembly comprises a cap axially pierced but in which some remaining matter needs to be pierced by the cannula to access the inside of the medicament bag, wherein the cap is initially tightly connected to the cannula.

The medicament delivery devices described herein can be used for the treatment and/or prophylaxis of one or more of many different types of disorders. Exemplary disorders include, but are not limited to: rheumatoid arthritis, inflammatory bowel diseases (e.g. Crohn's disease and ulcerative colitis), hypercholesterolaemia, diabetes (e.g. type 2 diabetes), psoriasis, migraines, multiple sclerosis, anaemia, lupus, atopic dermatitis, asthma, nasal polyps, acute hypoglycaemia, obesity, anaphylaxis and allergies. Exemplary types of drugs that could be included in the medicament delivery devices described herein include, but are not limited to, small molecules, hormones, cytokines, blood products, antibodies, antibody-drug conjugates, bispecific antibodies, proteins, fusion proteins, peptibodies, polypeptides, pegylated proteins, protein fragments, protein analogues, protein variants, protein precursors, chimeric antigen receptor T cell therapies, cell or gene therapies, oncolytic viruses, or immunotherapies and/or protein derivatives. Exemplary drugs that could be included in the medicament delivery devices described herein include, but are not limited to (with non-limiting examples of relevant disorders in brackets): etanercept (rheumatoid arthritis, inflammatory bowel diseases (e.g. Crohn's disease and ulcerative colitis)), evolocumab (hypercholesterolaemia), exenatide (type 2 diabetes), secukinumab (psoriasis), erenumab (migraines), alirocumab (rheumatoid arthritis), methotrexate (amethopterin) (rheumatoid arthritis), tocilizumab (rheumatoid arthritis), interferon beta-1a (multiple sclerosis), sumatriptan (migraines), adalimumab (rheumatoid arthritis), darbepoetin alfa (anaemia), belimumab (lupus), peginterferon beta-1a′ (multiple sclerosis), sarilumab (rheumatoid arthritis), semaglutide (type 2 diabetes, obesity), dupilumab (atopic dermatitis, asthma, nasal polyps, allergies), glucagon (acute hypoglycaemia), epinephrine (anaphylaxis), insulin (diabetes), atropine and vedolizumab (inflammatory bowel diseases (e.g. Crohn's disease and ulcerative colitis)), ipilimumab, nivolumab, pembrolizumab, atezolizumab, durvalumab, avelumab, cemiplimab, rituximab, trastuzumab, ado-trastuzumab emtansine, fam-trastuzumab deruxtecan-nxki, pertuzumab, transtuzumab-pertuzumab, alemtuzumab, belantamab mafodotin-blmf, bevacizumab, blinatumomab, brentuximab vedotin, cetuximab, daratumumab, elotuzumab, gemtuzumab ozogamicin, 90-Yttrium-ibritumomab tiuxetan, isatuximab, mogamulizumab, moxetumomab pasudotox, obinutuzumab, ofatumumab, olaratumab, panitumumab, polatuzumab vedotin, ramucirumab, sacituzumab govitecan, tafasitamab, or margetuximab. Pharmaceutical formulations including, but not limited to, any drug described herein are also contemplated for use in the medicament delivery devices described herein, for example pharmaceutical formulations comprising a drug as listed herein (or a pharmaceutically acceptable salt of the drug) and a pharmaceutically acceptable carrier. Pharmaceutical formulations comprising a drug as listed herein (or a pharmaceutically acceptable salt of the drug) may include one or more other active ingredients, or may be the only active ingredient present.

Exemplary drugs that could be included in the medicament delivery devices described herein include, but are not limited to, an immuno-oncology or bio-oncology medications such as immune checkpoints, cytokines, chemokines, clusters of differentiation, interleukins, integrins, growth factors, enzymes, signaling proteins, pro-apoptotic proteins, anti-apoptotic proteins, T-cell receptors, B-cell receptors, or costimulatory proteins.

Exemplary drugs that could be included in the medicament delivery devices described herein include, but are not limited to, those exhibiting a proposed mechanism of action, such as HER-2 receptor modulators, interleukin modulators, interferon modulators, CD38 modulators, CD22 modulators, CCR4 modulators, VEGF modulators, EGFR modulators, CD79b modulators, Trop-2 modulators, CD52 modulators, BCMA modulators, PDGFRA modulators, SLAMF7 modulators, PD-1/PD-L1 inhibitors/modulators, B-lymphocyte antigen CD19 inhibitors, B-lymphocyte antigen CD20 modulators, CD3 modulators, CTLA-4 inhibitors, TIM-3 modulators, VISTA modulators, INDO inhibitors, LAG3 (CD223) antagonists, CD276 antigen modulators, CD47 antagonists, CD30 modulators, CD73 modulators, CD66 modulators, CDw137 agonists, CD158 modulators, CD27 modulators, CD58 modulators, CD80 modulators, CD33 modulators, APRIL receptor modulators, HLA antigen modulators, EGFR modulators, B-lymphocyte cell adhesion molecule modulators, CDw123 modulators, Erbb2 tyrosine kinase receptor modulators, mesothelin modulators, HAVCR2 antagonists, NY-ESO-1 OX40 receptor agonist modulators, adenosine A2 receptors, ICOS modulators, CD40 modulators, TIL therapies, or TCR therapies.

Exemplary drugs that could be included in the medicament delivery devices described herein include, but are not limited to, a multi-medication treatment regimen such as AC, Dose-Dense AC, TCH, GT, EC, TAC, TC, TCHP, CMF, FOLFOX, mFOLFOX6, mFOLFOX7, FOLFCIS, CapeOx, FLOT, DCF, FOLFIRI, FOLFIRINOX, FOLFOXIRI, IROX, CHOP, R-CHOP, RCHOP-21, Mini-CHOP, Maxi-CHOP, VR-CAP, Dose-Dense CHOP, EPOCH, Dose-Adjusted EPOCH, R-EPOCH, CODOX-M, IVAC, HyperCVAD, R-HyperCVAD, SC-EPOCH-RR, DHAP, ESHAP, GDP, ICE, MINE, CEPP, CDOP, GemOx, CEOP, CEPP, CHOEP, CHP, GCVP, DHAX, CALGB 8811, HIDAC, MOPAD, 7+3, 5+2, 7+4, MEC, CVP, RBAC500, DHA-Cis, DHA-Ca, DHA-Ox, RCVP, RCEPP, RCEOP, CMV, DDMVAC, GemFLP, ITP, VIDE, VDC, VAI, VDC-IE, MAP, PCV, FCR, FR, PCR, HDMP, OFAR, EMA/CO, EMA/EP, EP/EMA, TP/TE, BEP, TIP, VIP, TPEx, ABVD, BEACOPP, AVD, Mini-BEAM, IGEV, C-MOPP, GCD, GEMOX, CAV, DT-PACE, VTD-PACE, DCEP, ATG, VAC, VelP, OFF, GTX, CAV, AD, MAID, AIM, VAC-IE, ADOC, or PE.

Exemplary drugs that could be included in the medicament delivery devices described herein include, but are not limited to, those used for chemotherapy, such as an alkylating agent, plant alkaloid, antitumor antibiotic, antimetabolite, or topoisomerase inhibitor, enzyme, retinoid, or corticosteroid. Exemplary chemotherapy drugs include, by way of example but not limitation, 5-fluorouracil, cisplatin, carboplatin, oxaliplatin, doxorubicin, daunorubicin, idarubicin, epirubicin, paclitaxel, docetaxel, cyclophosphamide, ifosfamide, azacitidine, decitabine, bendamustine, bleomycin, bortezomib, busulfan, cabazitaxel, carmustine, cladribine, cytarabine, dacarbazine, etoposide, fludarabine, gemcitabine, irinotecan, leucovorin, melphalan, methotrexate, pemetrexed, mitomycin, mitoxantrone, temsirolimus, topotecan, valrubicin, vincristine, vinblastine, or vinorelbine.

Furthermore, all terms used in the claims are to be interpreted according to their ordinary meaning in the technical field, unless explicitly defined otherwise herein. All references to “a/an/the element, apparatus, component, means, etc.” are to be interpreted openly as referring to at least one instance of the element, apparatus, component, means, etc., unless explicitly stated otherwise.

### BRIEF DESCRIPTION OF THE DRAWINGS

Embodiments of the inventive concept will now be described, by way of example only, with reference to the accompanying drawings, in which:

FIG. 1 schematically shows a medicament delivery device comprising a cannula assembly of the invention.

FIG. 2 schematically shows an infusion container configured to be used with the cannula assembly of the invention.

FIGS. 3-9 schematically show perspective views and cross-section views of the cannula assembly of the invention in different examples.

FIG. 10 schematically shows a reusable body of the medicament delivery device of FIG. 1 having multiple cannula assemblies of the invention.

### DETAILED DESCRIPTION

A medicament delivery system for delivering high volume/viscosity medicaments usually comprise infusion containers and infusion tubes. The infusion tube is configured to be attached to a medicament delivery member, e.g., an injection needle or a soft cannula that is configured to be embedded into the patient's body.

Furthermore, the medicament delivery system is preferred to have a medicament delivery device having a power source to automatically expel the medicament contained within the infusion container; the infusion tube; and the medicament delivery member. As most of the high volume/viscosity medicament deliveries take a long time to complete the medicament delivery operation, most of the medicament delivery devices are designed with wearable features so that the patients do not need to hold the medicament delivery device for long periods of time.

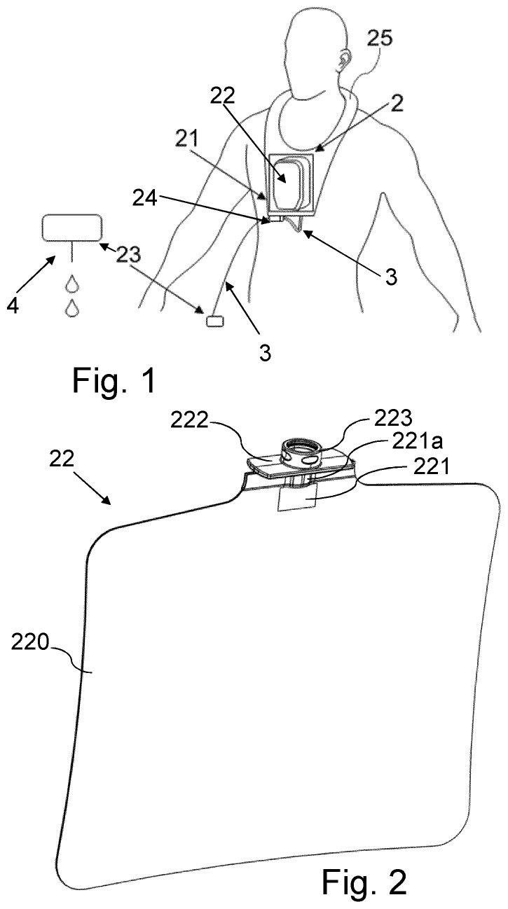

FIGS. 1-10 illustrate a medicament delivery device that can be used with multiple examples of cannula assemblies of the invention and multiple examples of cannula assemblies of the invention. In particular, FIG. 1 shows one example of the medicament delivery device 2 that can be used with the cannula assembly of the invention. The medicament delivery device 2 comprises a reusable body 21 configured to accommodate an infusion container 22; an infusion tube 3 is operably connected to the infusion container 22 and a medicament delivery member assembly 23 having a medicament delivery member 4; a wearable feature 25, e.g., a stripe, a belt, or a garment, extending from the reusable body and being configured to be worn by a patient. Furthermore, the medicament delivery device also comprises a power source 24 configured to output force to the infusion container to expel medicament contained within the infusion container 22 out from the medicament delivery member 4. The power source 24 is configured to deliver force over a long period of time, e.g., a couple of hours or days. For example, the power source 24 can be a motor, a fluid pressure power source, e.g., a gas pump or a hydraulic pump, or a peristaltic pump.

The cannula assembly of the invention is configured to be fluidly connected between an infusion container and an infusion tube so that the medicament contained within the infusion container 22 can be delivered to the infusion tube 3 for being delivered to the patient. The fluid connection between the cannula assembly, the infusion container 22, and the infusion tube 3 is configured to be established when the patient plans to start the medicament delivery operation so that the infusion container 22 can be in a sterilized state until the patient wants to receive the contained medicament.

FIG. 2 shows an example of the infusion container 22. The infusion container 22 comprises a body 220 with a fluid outlet 221. The body 220 is configured to accommodate medicament. The medicament contained within the body 220 is configured to be moved out of the body 220 through the fluid outlet 221. The body 220 comprises a flexible portion. In a preferred example, the body 220 comprises a flexible bag 220. The infusion container 22 comprises a connector 222, 223 attached to the body 220 and adjacent to the fluid outlet 221. The connector 222, 223 comprises a fastener 222 extending from the connected away from the body 220 of the infusion container 22 and configured to be operably connected to the infusion tube 3. A fluid channel 221a is provided in a body of the connector 222, 223 and lined up with the fluid outlet 221. The fluid channel 221a and/or port 223 of the connector 222, 223 may be sealed by a membrane and/or arranged with a one-way valve depending on the contained medicament preservation requirement.

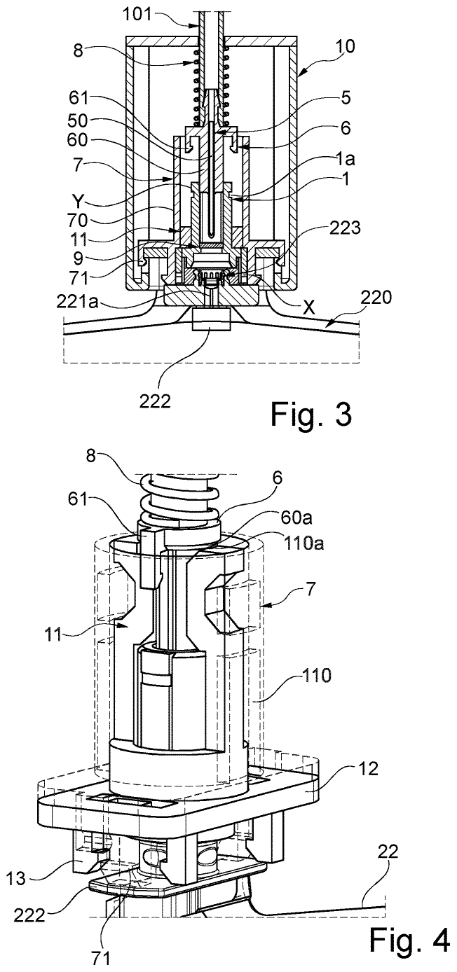

As shown in FIGS. 3-9, the cannula assembly of the invention comprises a housing 1 extending between a container end X configured to be operably attached to the infusion container 22 and a tube end Y configured to be operably attached to the infusion tube 3 along a longitudinal axis L; a cannula hub 6 movably arranged at least partially within the housing 1; and a cannula 5 comprising a cannula body 50 having a tube section configured to be operably attached to the infusion tube 3 and a container section configured to be operably attached to the infusion container 22. In one example, the cannular hub is fully positioned within the housing and movable relative to the housing 1. In this example, the risk of the user accidentally moves the cannular hub can be reduced. Alternatively, the cannular hub is partially position within the housing and movable relative to the housing.

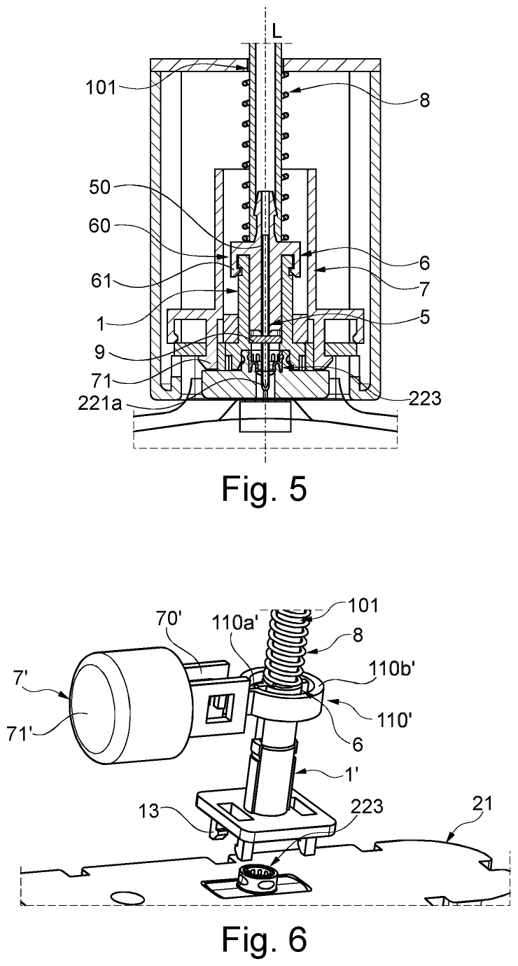

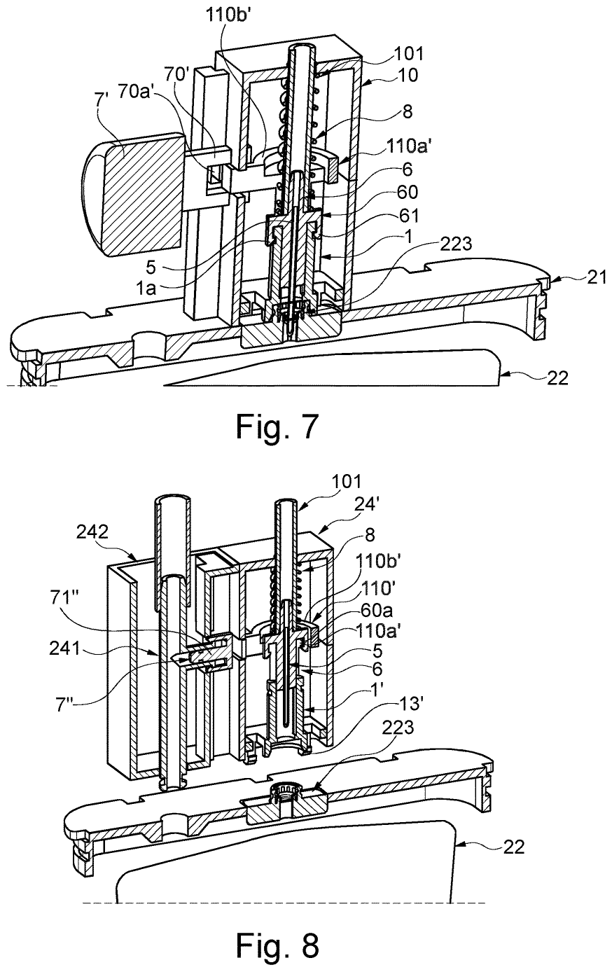

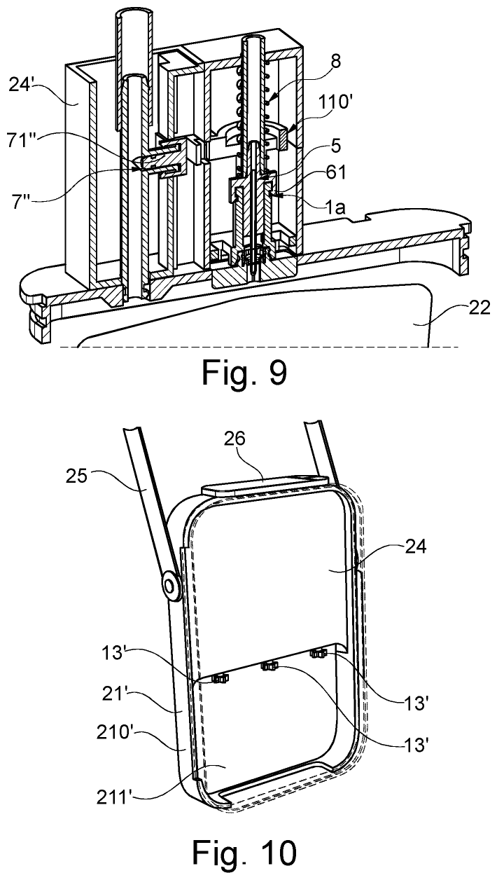

The container section of the cannula body 50 is attached to the cannula hub 6. In one example, both the tube section of the cannular body 50 and the container section of the cannular body 50 are attached to the cannular hub 6 as shown in FIG. 3. Alternatively, only the container section of the cannular body is attached to the cannular hub. The cannula hub 6 is movable relative to the housing 1 in the direction of the longitudinal axis L between an initial position where the container section of the cannula body 50 of the cannula 5 is fully received in the housing 1, as shown in FIG. 3 and FIG. 8, and a connected position where the cannula body 50 of the cannula 5 is positioned at least partially outside of the container end X of the housing 1, as shown in FIG. 5, FIG. 7, and FIG. 9.

The container section of the cannula body 50 comprises a sharp end for opening a seal of the infusion container 22. The tube section, on the other hand, doesn't need to be sharp as the tube section is only configured to be connected to the infusion tube 3, not piercing patient's skin or a seal.

The cannula hub 6 comprises a fluid channel being coaxial with the cannula body 50; and the tube section of the cannula body 50 is fully received within the cannula hub 6, as shown in FIG. 3.

When the cannula assembly is attached to the infusion container, the container end X of the housing 1 is adjacent to the infusion container, as shown in FIG. 3; as a result, once the cannula 5 is positioned outside of the container end X of the housing 1, the fluid connection between the infusion container 22 and the cannula 5 is established. Preferably, when the cannula assembly is attached to the infusion container 22 and the cannula hub 6 is in the connected position, the cannula 5 is snuggly fitted into the fluid channel 221a.

In a preferred example, the cannula 5 comprises a sharp end at the container section. The sharp end of the cannula 5 is configured to face towards the fluid channel 221a of the infusion container such that if the fluid channel 221a is sealed by a membrane, the cannula 5 can pierce the membrane before the fluid connection between the cannula body 50 and the infusion container 22 is established. Furthermore, in a preferred example, the container section of the cannula is sealed by a sheath. Alternatively, the container end of the housing is sealed by a membrane 9, as shown in FIG. 3.

The container end of the housing of the cannula assembly is configured to be attached to the infusion container, e.g., by being glued or tied with tape. In one example where the connector 222, 223 of the infusion container 22 comprises the fastener 222, the housing 1 comprises a container fastener 13 configured to be operably attached to the infusion container 22. The container fastener 13 is nearby the container end X of the housing 1. The container fastener 13 is configured to be attached to the fastener 222 of the connector 222, 223 of the infusion container 22. In a preferred example, the container fastener 13 is at least one of a thread, a bayonet connector, and a snap-fit connector. For example, as shown in FIG. 4, the container fastener 13 is a snap-fit arm extending from the container end X of the housing 1 in the direction of the longitudinal axis L, thus, a patient and/or a caregiver, e.g., a nurse or a pharmacist can be easily attached the cannula assembly to the infusion container 22.

In a preferred example, the housing 1 comprises a hub fastener 1a and a cannula hub 6 comprises a counter fastener 61 configured to be attached to the hub fastener 1a of the housing 1 when the cannula hub 6 is in the connected position such that the cannula 5 is immovable relative to the infusion container 22 during the medicament delivery operation. In a preferred example, the hub fastener 1a of the housing 1 and the counter fastener 61 of the cannula hub 6 are configured to form a snap-fit attachment when the cannula hub 6 is in the connected position. For example, the counter fastener 61 of the cannula hub 6 comprises a snap-fit arm 61 with a hook at its free end and the snap-fit arm extending from a wall 60 of the cannula hub 6 in the direction transverse to the longitudinal axis L. In this example, the hub fastener 1a of the housing 1 is a recess/cut-out in a wall of the housing 1.

Furthermore, in one example, the housing 1 comprises an outer shell 10 configured to enclose the cannula hub 6. In one example, the outer shell 10 comprises a tube connector 101 extending from a portion of the cannula hub 6 in which the tube section of the cannula 5 is placed.

The tube connector 101 is configured to be attached to the infusion tube 3. In one example, the infusion tube can be attached to the outer shell 10 of the housing 1 of the cannula assembly before being delivered to the patient and/or the caregiver. Alternatively, the infusion tube 3 and the infusion container 22 can be delivered independently. The patient and/or the caregiver will receive the infusion tube 3, the cannula assembly and the infusion container 22 as three isolated components and will need to connect the infusion tube 3 to the cannula assembly. Alternatively, the infusion tube 3 and the cannular assembly can be preassembled together before delivered to the patient and/or the caregiver.

As the cannula hub 6 and the cannula 5 are attached to one another, the cannula hub 6 can move the cannula 5 into the fluid channel 221a when the cannula hub 6 is in the connected position. In one example, the patient and/or the caregiver can manually move the cannula hub from the initial position to the connected position. In a preferred example, the cannula hub comprises a protruding section protruding from the housing such that the cannula hub is configured to be moved from the initial position to the connected position manually by a user by moving the protruding section relative to the housing. For example, the protruding section of the cannula hub is positioned through a longitudinally extending slot in a wall of the housing. In a preferred example, the protruding section of the cannula hub extends in a direction transverse to the longitudinal axis L away from the housing.

Alternatively, the cannula hub can be moved from the initial position to the connected position automatically when the patient and/or the caregiver starts the medicament delivery operation or intentionally triggers the connection between the cannula and the infusion container. In this example, the manual connection errors can be avoided. In this example, the cannula assembly comprises a biasing member 8 extending in the direction of the longitudinal axis L between a surface of the housing and a surface of the cannula hub 6 such that the cannula hub is biased towards the connected position by the biasing member 8. In one example where the housing 1 comprises the outer shell 10, the biasing member 8 is extending between a surface of the outer shell 10 that is facing towards the container end X of the housing 1 and a surface of the cannula hub that is facing away from the container end X of the housing 1, as shown in FIGS. 3, 5 and 7-9. In a preferred example, the biasing member 8 is a spring, e.g., a compression spring, a tension spring, or a torsion spring.

The cannula assembly further comprises a cannula hub lock 110; 110′ comprises a surface 110a; 110a′ facing away from the container end X of the housing 1. The cannula hub 6 comprises a counter surface 60a directed towards the container end X of the housing 1. The cannula hub lock 110; 110′ is immovable relative to the housing 1 in the direction of the longitudinal axis L. In one example, the cannula hub lock is an integral part of the housing. Alternatively, the cannula hub lock is attached to the housing via a thread connection, a bayonet connection, or a snap-fit connection. Alternatively, a ledge extends from the wall of the housing towards the longitudinal axis; and the cannula hub lock is placed on the ledge. Alternatively, a recess/cut-out is arranged in the wall of the housing; and the cannula hub lock is placed within the recess/cut-out of the housing.

The surface 110a; 110a′ of the cannula hub lock 110; 110′ is engaged with the counter surface 60a of the cannula hub 6 in a locked position, as shown in FIG. 3 and FIG. 8 such that the cannula hub 6 is prevented from being biased into the connected position and is disengaged from the counter surface of the cannula hub in a released position, as shown in FIG. 5, FIG. 7, and FIG. 9, such that the cannula hub 6 is biased into the connected position by the biasing member 8.

In a preferred example, the surface 110a; 110a′ of the cannula hub lock 110; 110′ is movable from the locked position to the released position in the direction transverse to the longitudinal axis L.

Preferably, the cannula assembly comprises a lock holder 7; 7′; 7″ having a body movable relative to the housing 1. The body of the lock holder 7; 7′; 7″ comprises an inner section 70; 70′ positioned within the housing 1 and an outer section 71; 71′; 71″ positioned outside of the housing 1. The cannula hub lock 110; 110′ is operably connected to the lock holder 7; 7′; 7″.

In another preferred example, the surface 110a; 110a′ of the cannula hub lock 110; 110′ is radially flexible from the locked position to the released position relative to the longitudinal axis L. In one example, the cannula hub lock 110 comprises a flexible arm 11 extending from the housing 1. The flexible arm 11 comprises a ledge having the surface 110a of the cannula hub lock 110. In one example the lock holder 7 is configured to move in the direction of the longitudinal axis L relative to the housing 1. In one example, the lock holder 7 is an outer sleeve 7 movable relative to the housing 1 in the direction of the longitudinal axis L between a first position where the outer sleeve 7 is circumferentially adjacent to the flexible arm 11, as shown in FIG. 3 and FIG. 4, and a second position where the outer sleeve 7 is circumferentially offset from the flexible arm 11. In a preferred example, the outer part 71 of the body of the lock holder 7 is a protrusion 71 extending beyond the container end X of the housing 1. In one example where the housing 1 comprises the container fastener 13, the protrusion 71 extends beyond the container fastener 13, as shown in FIG. 4. As a result, when the container end X of the housing 1 is attached to the infusion container 22, the infusion container 22 pushes the outer sleeve 7 away via the protrusion 71. Thus, the outer sleeve 7 is moved relative to the housing 1 in the longitudinal axis L from the first position to the second position such that the flexible arm 11 can flex radially outward. In one example where the protrusion 71 extends beyond the container fastener 13 of the housing 1, when the container fastener 13 of the housing 1 is attached to the fastener 222 of the infusion container 22, the protrusion 71 is pushed to move relative to the housing 1 by a portion of the fastener 222 of the infusion container 22, as shown in FIG. 4. In one example, an interface formed between the surface 110a of the cannula hub lock 110 and the counter surface 60a of the cannula hub 6 is a chamfered surface such that once the outer sleeve 7 is in the second position, the flexible arm 11 is flexed radially outward by the biasing member 8 via counter surface 60a of the cannula hub 6. Alternatively, or additionally, the flexible arm is pre-tensioned inward to the longitudinal axis L when the outer sleeve is in the first position.

Once the flexible arm 11 is flexed radially outward, the surface 110a of the cannula hub lock 110 is disengaged from the counter surface 60a of the cannula hub 6 (the cannula hub lock 110 is moved into the released position); thus, the cannula hub 6 is biased to the connected position by the biasing member 8, as shown in FIG. 5. In a preferred example, the when the cannula hub 6 is moved into the connected position, the counter fastener 61 of the cannula hub 6 is snap-fitted into the hub fastener 1a of the housing 1.

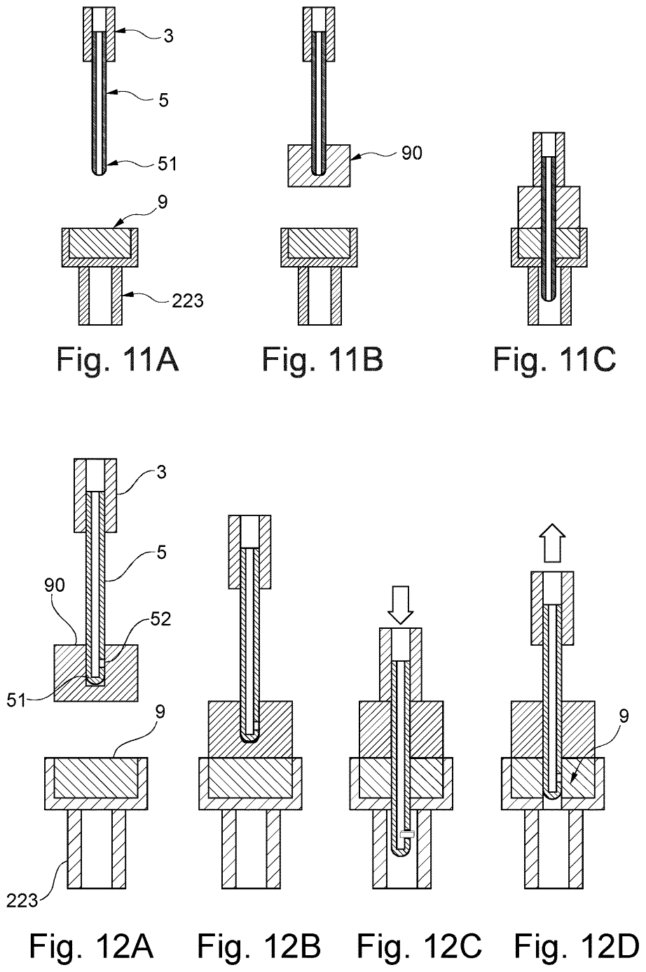

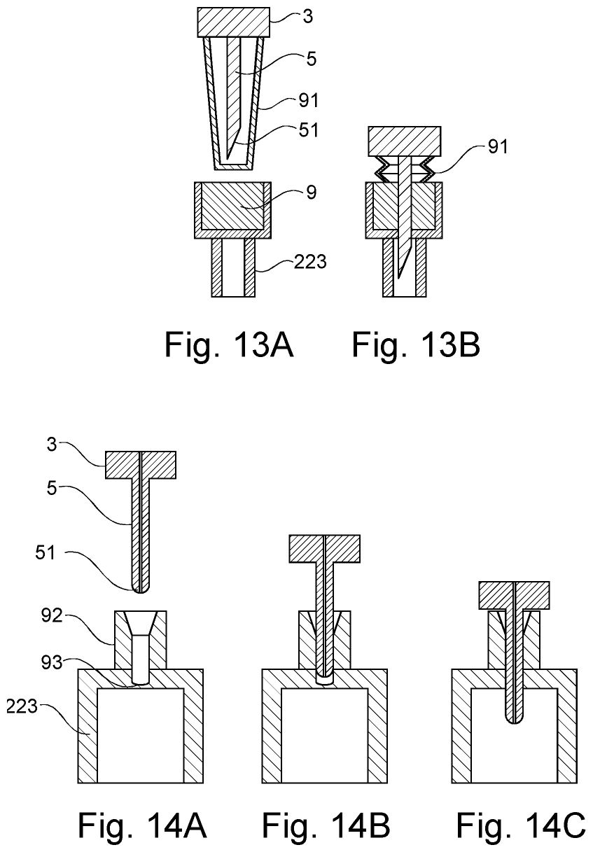

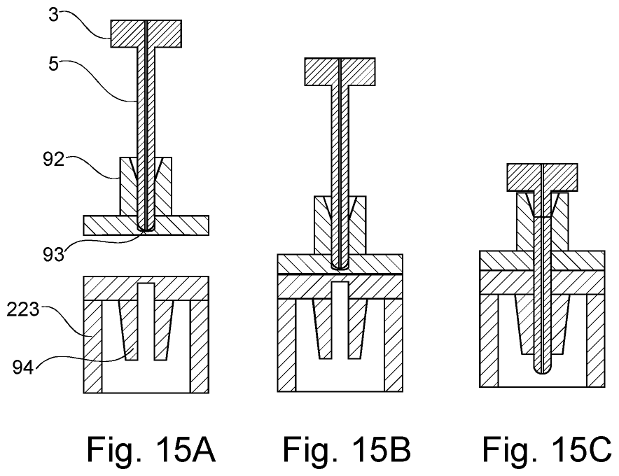

In this example, the fluid connection between the cannula assembly and the infusion container 22 is established once the patient and/or the caregiver attach the cannula assembly to the infusion container 22. For example, the cannula assembly can be delivered to the patient as an independent component or can be arranged within the reusable body 21′ of the medicament delivery device as shown in FIG. 10. In a preferred example, the cannular assembly is removably arranged within the reusable body 21′. For example, the cannular assembly can be immovably attached to the connector 222, 223 of the infusion container 22; alternatively, the container fastener of the housing is configured to be immovably attached to the container. As a result, the cannular assembly can be removed together with the infusion container 22 away from the reusable body 21′ after use.

Alternatively, the surface of the cannula hub lock is linearly movable between the locked position and the released position in the direction transverse to the longitudinal axis L. Alternatively, the surface of the cannula hub lock is pivotal from the locked position to the released position relative to the longitudinal axis L.

Alternatively, in another example, the cannula hub lock 110′ comprises a clip having a clip body 110b′ enclosing the cannula hub 5. The clip body 110b′ comprises a ledge having the surface 110a′ of the cannula hub lock 110′. The clip body 110b′ is circumferentially expandable relative to the longitudinal axis L such that the surface 110a′ of the cannula hub lock 110′ is moved from the lock position to the released position when the clip body 110b′ is circumferentially expanded outwards from the longitudinal axis L. Similar to the above-mentioned example, the clip body can be configured to be engaged with/disengaged from the outer sleeve such that the axial movement of the outer sleeve is configured to allow the cannula hub lock to move into the released position. Similar to the above-mentioned example, an interface formed between the surface 110a′ of the clip body 110b′ and the counter surface of the cannula hub 6 is a chamfered surface such that once the outer sleeve is disengaged from the clip body, the clip body is expanded outward by the biasing member 8 via the counter surface of the cannula hub 6. Alternatively, or additionally, the clip body is pre-tensioned inward to the longitudinal axis L when the outer sleeve is in the first position.

Alternatively, in another example, the lock holder 7′; 7″ of the cannula assembly is configured to move in the direction transverse to the longitudinal axis L. In one example where the cannula hub lock 110 comprises the flexible arm 11 as mentioned above, the inner section of the lock holder is configured to surround the flexible arm 11 such that the cannula hub lock cannot move to the released position until the inner section of the lock holder is spaced apart from the flexible arm of the cannula hub lock by being moved in the direction transverse to the longitudinal axis L via the outer section 71′ of the body.

In one example where the cannula hub lock 110′ comprises the clip, the lock holder 7′; 7″ is movable relative to the clip from a first position where the inner section 70′ of the body of the lock holder 7′ is coupled to the clip such that the clip body 110b′ is prevented from expanding to a second position where the inner section 70′ of the body of the lock holder is decoupled from the clip such that the clip body 110b′ is allowed to expand. For example, the inner section of the body is engaged with the clip body when the inner section of the body of the lock holder is coupled to the clip. The inner section of the body lock holder is disengaged from the clip body when the inner section of the body is decoupled from the clip. Alternatively, or additionally, the cannula hub lock 110; 110′ comprises two extensions extending in the direction transverse to the longitudinal axis L. The two extensions are configured to be spaced apart from one another. The clip body 110b′ is extending in a circumferential direction around the longitudinal axis from one of the two extensions to the other one of the two extensions. The inner section 70′ of the body of the lock holder 7′; 7″ is engaged with the two extensions when the inner section of the body is coupled to the clip such that the two extensions are pushed towards one another; and wherein the inner section 70′ of the body of the lock holder 7′ is disengaged from the extensions when the inner section 70′ of the body of the lock holder 7′ is decoupled from the clip. For example, cut-outs 70a′ are arranged in walls of the inner section 70′ of the body of the lock holder 7′; 7″, as shown in FIGS. 6-7. As a result, the walls of the inner section 70′ is engaged with the extensions of the clip, thus the clip body 110b′ cannot expand to the released position until the lock holder 7′ is moved in the direction transverse to the longitudinal axis L relative to the clip, the cut-outs are moved to be aligned with the extensions, thus the clip body 110b′ can expand circumferentially and move the extensions into the cut-outs; thus, the cannula hub lock is moved into the released position.

In one example, as shown in FIGS. 6-7, the outer section 71′ of the body of the lock holder 7′ is a user-accessible button such that the lock holder 7′ is moved by a user of the cannula assembly manually to cause the cannula hub lock to be moved from the locked position to the released position. For example, the patient and/or the caregiver can press the button to establish the fluid connection between the infusion container 22 and the cannula 5 when the infusion tube 3 is connected, and the patient is ready to receive the medicament.

Alternatively, the outer section 71″ of the body of the lock holder 7″ is operably connected to the medicament delivery device such that the movement of the lock holder 7″ is initiated by a mechanism of the medicament delivery device. In a preferred example, the outer section 71″ of the body of the lock holder″ is operably connected to the power source 24 of the medicament delivery device. In one example, the outer section 71″ of the body of the lock holder 7″ is configured to be positioned within a gas-tight chamber 241 such that the lock holder 7″ is moved by a change of the pneumatic pressure within the gas-tight chamber 241 to cause the cannula hub lock 110′ to be moved from the locked position to the released position, as shown in FIGS. 9-10. In this shown example, the inner section of the body of the lock holder 7″, and the clip of the cannula hub lock 110′ are the same as the previous example. Preferably, the outer section 71″ of the body of the lock holder 7″ is a pin gas-tightly positioned in a channel that is fluidly connected to the gas-tight chamber 241. In this example, the medicament delivery device comprises a pneumatic actuation mechanism connected to the gas-tight chamber 241. In a preferred example, the medicament delivery device comprises a pneumatic power source 24, e.g., a gas pump, comprising a gas outlet 242 connected to the gas-tight chamber 241. In one example, the gas pump is configured to create a vacuum in eh gas-tight chamber 241 such that the outer section 71′ of the body of the lock holder 7″ is pulled and the lock holder 7″ is moved into the second position. Alternatively, the gas pump can deliver gas into the gas-tight chamber; as a result, the lock holder 7″ is pushed to the second position. In this example, the cannula assembly can be mainly invisible to the user, e.g., patients and/or caregivers, as shown in FIG. 10. For example, the reusable body 21′ of the medicament delivery device 2 is configured to receive multiple infusion containers. In this example, the reusable body 21′ of the medicament delivery device 2 comprises a functional section to receive the power source and the cannula assemblies, and a medicament section 210′ to receive the infusion containers. For example, the medicament section 210′ can be covered by a lid 211′, the user can open the lid 211′ can attach the infusion containers to the cannula assemblies respectively via the fastener 222 of each infusion container 22 and the container fastener 13 of each cannula assemblies.

In this example, the medicament delivery device comprises a control interface 26, e.g., a touch panel or buttons, to control the establishment of the fluid connection between each infusion container and each cannula.

For example, the patient might need to take different medicaments in a specific order. In this example, the infusion tube comprises branches connected to housings of the cannula assemblies respectively. The power source 24 is configured to initiate the fluid connection between one specific cannula and one specific infusion container when the medicament contained within that specific infusion container is ready to be delivered. In one example where the infusion container is a flexible bag, and the power source of the medicament delivery device is a pneumatic power source, the medicament delivery device can be operated to control the sequence of the medicament delivery by controlling the sequence of establishing the fluid connection between the cannula assembly and the infusion container. For example, the medicament delivery device can comprise three infusion containers containing three different medicaments: A, B, and C respectively, the patient is prescribed to have a treatment in the following steps with the following orders:
1. taking A medicament;
2. taking B medicament;
3. stopping any medicament intake for 15 minutes; and
4. finally taking C medicament.

In this example, the pneumatic power source 24 of the medicament delivery device can operate in the following steps with the following orders:
1. moving the outer section 71″ of the body of the lock holder 7′ that is in the cannula assembly connected to the infusion container with A medicament such that the canular lock 110′ is moved into the released position and the cannula hub 6 is moved into the connected position, thus the fluid connection between the cannula assembly connected to the infusion container with A medicament is established;
2. delivering the gas into the medicament section of the reusable body to expel A medicament to the patient until the A medicament is fully delivered;
3. establishing the fluid connection between the cannula assembly connected to the infusion container with B medicament is established by the method as defined in step 1;
4. expelling B medicament to the patient by the method as defined in step 2;
5. counting down for 15 minutes;
6. establishing the fluid connection between the cannula assembly connected to the infusion container with C medicament is established by the method as defined in step 1; and
7. expelling C medicament to the patient by the method as defined in step 2.

Alternatively, the infusion containers are positioned in multiple gas-tight chambers respectively such that the medicament delivery rate of each medicament can be controlled independently.

Alternatively, the medicament delivery device comprises only one infusion container; in this example, the power source of the medicament delivery device is configured to initiate the fluid connection between one cannula assembly and the one infusion container.

To maintain a sterile barrier for the cannula 5 after sterilization, the cannula 5 may be hermetically enclosed in a chamber. On FIG. 3, such a chamber is delimited on one side by the soft membrane 9. The membrane 9 is a flexible rubberlike membrane, and can be pierced by the cannula 5. The chamber is delimited on other sides by the housing 1 and the needle hub 6. This requires an airtight connexion, that is to say a sealed interface, between the needle hub 6 and the housing 1.

That design may be simplified by reducing sealed interfaces, as shown on FIGS. 11B and 11C. FIG. 11A shows a situation where the cannula 5 is not enclosed in a sterile chamber. One end of the cannula 5 is connected to the infusion tube 3. On the opposite end, the tjp 51 of the cannula 5 is facing the soft membrane 9 attached to the bag port 223. Fig. B shows a modified embodiment in which a penetrable cap membrane 90 is tightly connected to the tip 51 of the cannula 5, forming a cap around the tip 51 of the cannula 5, before the soft membrane 9 is pierced. The penetrable cap membrane 90 is a soft, flexible, rubberlike membrane that can be pierced. During the piercing movement, the penetrable cap membrane 90 comes in contact with the soft membrane 9, then the tip 51 of the cannula 5 pierces the penetrable cap membrane 90 and then the soft membrane 9, and the penetrable cap membrane 90 is pushed towards the infusion tube 3. The configuration at the end of the piercing movement is shown on FIG. 11C.

The embodiment of FIGS. 11B and 11C may be modified by using a cannula 5 with a blunt tip 51 and a side opening 52, as shown on FIG. 12A to 12D. This design adds a resealable function to the cannula 5, similar to a valve. FIG. 12A shows the configuration before the piercing occurs. FIG. 12B shows the configuration when the penetrable cap membrane 90 comes in contact with the soft membrane 9. FIG. 12C shows the configuration after the tip 51 of the cannula 5 has pierced the penetrable cap membrane 90 and then the soft membrane 9. FIG. 12D shows the configuration once the cannula 5 has been retracted, that is to say brought back out of the medicament bag. In that embodiment, the cannula can be retracted and sealed off in the soft membrane 9.

The embodiment of FIG. 11A can be modified by using a compressible membrane 91, as shown on FIG. 13A. The compressible membrane 91 is a soft, flexible, rubberlike membrane that can be pierced and compressed. The compressible membrane 91 covers the cannula 5 and creates a sterile barrier around the cannula 5 prior use. During the piercing movement, the tip 51 of the cannula 5 pierces the soft membrane 9, which compresses the compressible membrane 91, as shown on FIG. 13B.

The embodiments of FIGS. 11A to 13B show a design with a soft rubberlike membrane 9 to pierce through and/or to seal again the cannula 5. That membrane 9 may be assembled to the bag port 223 by pushfit, welding, glueing, or overmolding during injection molding.

However instead of the membrane 9, the bag port 223 may comprise a cap 92, as shown on FIG. 14A. Said cap 92 is axially pierced but the piercing does not go through to the inside of the medicament bag, some remaining matter 93 needs to be pierced by the cannula 5 to access the inside of the medicament bag. FIG. 14A shows the initial configuration, FIG. 14B shows the configuration just before the cannula pierces the remaining matter 93, and FIG. 14C shown the configuration at the end of the piercing movement.

In an alternative embodiment, the cap 92 is not part of the bag port 223, but a separate element. The cap 92 is initially mounted to the cannula 5, as shown on FIG. 15A. During the piercing movement, the cap 92 comes in contact with the bag port 223 (as shown on FIG. 15B), then the cannula 5 pierces the remaining matter 93, and the cap 92 is pushed towards the infusion tube 3. The configuration at the end of the piercing movement is shown on FIG. 15C. In that embodiment, the bag port 223 comprises a guide portion 94 configured to guide the cannula 5 once the cannula 5 has pierced the remaining matter 93 and is penetrating inside the bag.

The inventive concept has mainly been described above with reference to a few examples. However, as is readily appreciated by a person skilled in the art, other embodiments than the ones disclosed above are equally possible within the scope of the inventive concept, as defined by the appended claims.

Some other aspects of the invention are defined in the following clauses.

1. A cannula assembly of a medicament delivery system, the cannula assembly for being fluidly connected between an infusion container and an infusion tube; the cannula assembly comprising:
a housing extending between a container end configured to be operably attached to the infusion container and a tube end configured to be operably attached to the infusion tube along a longitudinal axis;
a cannula hub movably arranged at least partially within the housing;
a cannula comprising a cannula body having a tube section configured to be operably attached to an infusion tube and a container section configured to be operably attached to an infusion container;
wherein the container section of the cannula body is attached to the cannula hub; and
wherein the cannula hub is movable relative to the housing in the direction of the longitudinal axis between an initial position where the container section of the cannula body of the cannula is fully received in the housing and a connected position where the cannula body of the cannula is positioned at least partially outside of the container end of the housing.

2. The cannula assembly according to clause 1, wherein the cannula hub comprises a protruding section protruding from the housing such that the cannula hub is configured to be moved from the initial position to the connected position manually by a user by moving the protruding section relative to the housing.

3. The cannula assembly according to clause 2, wherein the protruding section of the cannula hub is positioned through a longitudinally extending slot in a wall of the housing.

4. The cannula assembly according to clause 1, comprising
a biasing member extending in the direction of the longitudinal axis between a surface of the housing and a surface of the cannula hub such that the cannula hub is biased towards the connected position by the biasing member; and
a cannula hub lock comprising a surface facing away from the container end of the housing;
wherein the cannula hub comprises a counter surface directed towards the container end of the housing; wherein the cannula hub lock is immovable relative to the housing in the direction of the longitudinal axis;
wherein the surface of the cannula hub lock is engaged with the counter surface of the cannula hub in a locked position such that the cannula hub is prevented from being biased into the connected position and is disengaged from the counter surface of the cannula hub in a released position such that the cannula hub is biased into the connected position by the biasing member.

5. The cannula assembly according to clause 4, wherein the cannula assembly comprises a lock holder having a body movable relative to the housing between a first position and a second position; wherein the body of the lock holder comprises an inner section positioned within the housing and an outer section positioned outside of the housing; and wherein the cannula hub lock is operably connected to the lock holder when the lock holder is in the first position and is not connected to the lock holder when the lock holder is in the second position.

6. The cannula assembly according to clause 4 or 5, wherein the biasing member is a spring.

7. The cannula assembly according to clauses 4-6, wherein the surface of the cannula hub lock is movable from the locked position to the released position in the direction transverse to the longitudinal axis.

8. The cannula assembly according to clause 7, wherein the surface of the cannula hub lock is radially flexible from the locked position to the released position relative to the longitudinal axis.

9. The cannula assembly according to clause 8 when dependent in clause 5, wherein the cannula hub lock comprises a flexible arm extending from the housing; wherein the flexible arm comprises a ledge having the surface of the cannula hub lock; and wherein the lock holder comprises an outer sleeve movable relative to the housing in the direction of the longitudinal axis between a first position where the outer sleeve is circumferentially adjacent to the flexible arm and a second position where the outer sleeve is circumferentially offset from the flexible arm.

10. The cannula assembly according to clause 9, wherein the outer section of the lock holder extends in the direction of the container end of the housing and beyond the container end of the housing in the direction of the longitudinal axis such that the outer sleeve is moved by the infusion container from the first position to the second position when the container end of the housing is attached to the infusion container.

11. The cannula assembly according to any one of clauses 4-8, wherein the cannula hub lock comprises a clip having a clip body enclosing the cannula hub; wherein the clip body comprises a ledge having the surface of the cannula hub lock; and wherein the clip body is circumferentially expandable relative to the longitudinal axis L such that the surface of the cannula hub lock is moved from the lock position to the released position when the clip body is circumferentially expanded outwards from the longitudinal axis.

12. The cannula assembly according to clause 11 when dependent on clause 5, wherein the lock holder is movable relative to the clip from a first position where the inner section of the body of the lock holder is coupled to the clip such that the clip body is prevented from expanding to a second position where the inner section of the body of the lock holder is decoupled from the clip such that the clip body is allowed to expand.

13. The cannula assembly according to clause 12, wherein the inner section of the body is engaged with the clip body when the inner section of the body of the lock holder is coupled to the clip; and wherein the inner section of the body of the lock holder is disengaged from the clip body when the inner section of the body is decoupled from the clip.

14. The cannula assembly according to clause 12, wherein the cannula hub lock comprises two extensions extending in the direction transverse to the longitudinal axis; wherein the two extensions are configured to be spaced apart from one another; wherein the clip body is extending in a circumferential direction around the longitudinal axis from one of the two extensions to the other of the two extensions; wherein the inner section of the body is engaged with the two extensions when the inner section of the body is coupled to the clip; and wherein the inner section of the body is disengaged from the extensions when the inner section of the body is decoupled from the clip.

15. The cannula assembly according to any one of clauses 5-14, wherein the outer section of the body of the lock holder is a user-accessible button such that the lock holder is moved by a user of the cannula assembly manually to causes the cannula hub lock to be moved from the locked position to the released position.

16. The cannula assembly according to any one of clauses 5-14, wherein the outer section of the body of the lock holder is configured to be positioned within a gas-tight chamber such that the lock holder is moved by a change of the pneumatic pressure within the gas-tight chamber to cause the cannula hub lock to be moved from the locked position to the released position.

17. The cannula assembly according to any one of the preceding clauses, the housing comprises a container fastener configured to be operably attached to the infusion container; wherein the container fastener is nearby the container end of the housing; and wherein the container fastener is at least one of a thread, a bayonet connector, and a snap-fit connector.

18. The cannula assembly according to any one of the preceding clauses, wherein the container section of the cannula is sealed by a sheath.

19. The cannula assembly according to any one of the preceding clauses, wherein the container end of the housing is sealed by a membrane.

20. The cannula assembly according to any one of the preceding clauses, wherein the housing comprises a hub fastener; wherein the cannula hub comprises a counter fastener attached to the hub fastener of the housing when the cannula hub is in the connected position.

21. The cannula assembly according to clause 20, wherein the hub fastener of the housing and the counter fastener of the cannula hub are configured to form a snap-fit attachment when the cannula hub is in the connected position.

22. The cannula assembly according to any of the previous clauses, comprising a penetrable cap membrane tightly connected to a tip of the cannula, forming a cap around the tip of the cannula, before a soft membrane sealing the container end of the housing is pierced.

23. The cannula assembly according to the previous clause, wherein the cannula has a blunt tip and a side opening.

24. The cannula assembly according to clauses 22 or 23, wherein the penetrable cap membrane is a compressible membrane covering the cannula and creating a sterile barrier around the cannula before a soft membrane sealing the container end of the housing is pierced.

25. The cannula assembly according to any of clauses 1 to 21, wherein a bag port comprises a cap axially pierced but in which some remaining matter needs to be pierced by the cannula (5) to access the inside of the medicament bag.

26. The cannula assembly according to any of clauses 1 to 21, comprising a cap axially pierced but in which some remaining matter needs to be pierced by the cannula to access the inside of the medicament bag, wherein the cap is initially tightly connected to the cannula.

1-15. (canceled)

16. A cannula assembly of a medicament delivery system, the cannula assembly for being fluidly connected between an infusion container and an infusion tube, the cannula assembly comprising:
a housing extending between a container end configured to be operably attached to the infusion container and a tube end configured to be operably attached to the infusion tube along a longitudinal axis;
a cannula hub movably arranged at least partially within the housing;
a cannula comprising a cannula body having a tube section configured to be operably attached to an infusion tube and a container section configured to be operably attached to an infusion container, wherein the container section of the cannula body is attached to the cannula hub, and wherein the cannula hub is movable relative to the housing in the direction of the longitudinal axis between an initial position where the container section of the cannula body of the cannula is fully received in the housing and a connected position where the cannula body of the cannula is positioned at least partially outside of the container end of the housing;
a biasing member extending in the direction of the longitudinal axis between a surface of the housing and a surface of the cannula hub such that the cannula hub is biased towards the connected position by the biasing member; and
a cannula hub lock comprising a surface facing away from the container end of the housing,
wherein the cannula hub comprises a counter surface directed towards the container end of the housing,
wherein the cannula hub lock is immovable relative to the housing in the direction of the longitudinal axis, and
wherein the surface of the cannula hub lock is engaged with the counter surface of the cannula hub in a locked position such that the cannula hub is prevented from being biased into the connected position and is disengaged from the counter surface of the cannula hub in a released position such that the cannula hub is biased into the connected position by the biasing member.

17. The cannula assembly of claim 16, wherein the cannula assembly comprises a lock holder having a body movable relative to the housing between a first position and a second position, wherein the body of the lock holder comprises an inner section positioned within the housing and an outer section positioned outside of the housing, and wherein the cannula hub lock is operably connected to the lock holder when the lock holder is in the first position and is not connected to the lock holder when the lock holder is in the second position.

18. The cannula assembly of claim 16, wherein the biasing member is a spring.

19. The cannula assembly of claim 16, wherein the surface of the cannula hub lock is movable from the locked position to the released position in the direction transverse to the longitudinal axis.

20. The cannula assembly of claim 19, wherein the surface of the cannula hub lock is radially flexible from the locked position to the released position relative to the longitudinal axis.

21. The cannula assembly of claim 20, wherein the cannula hub lock comprises a flexible arm extending from the housing, wherein the flexible arm comprises a ledge having the surface of the cannula hub lock, and wherein the lock holder is an outer sleeve movable relative to the housing in the direction of the longitudinal axis between a first position where the outer sleeve is circumferentially adjacent to the flexible arm and a second position where the outer sleeve is circumferentially offset from the flexible arm.

22. The cannula assembly according to claim 21, wherein the outer section of the lock holder extends in the direction of the container end of the housing and beyond the container end of the housing in the direction of the longitudinal axis such that the outer sleeve is moved by the infusion container from the first position to the second position when the container end of the housing is attached to the infusion container.

23. The cannula assembly of claim 16, wherein the cannula hub lock comprises a clip having a clip body enclosing the cannula hub, wherein the clip body comprises a ledge having the surface of the cannula hub lock, and wherein the clip body is circumferentially expandable relative to the longitudinal axis such that the surface of the cannula hub lock is moved from the lock position to the released position when the clip body is circumferentially expanded outwards from the longitudinal axis.

24. The cannula assembly of claim 23, wherein the lock holder is movable relative to the clip from a first position where the inner section of the body of the lock holder coupled to the clip such that the clip body is prevented from expanding to a second position where the inner section of the body of the lock holder is decoupled from the clip such that the clip body is allowed to expand.

25. The cannula assembly of claim 24, wherein the inner section of the body is engaged with the clip body when the inner section of the body of the lock holder is coupled to the clip, and wherein the inner section of the body of the lock holder is disengaged from the clip body when the inner section of the body is decoupled from the clip.

26. The cannula assembly of claim 24, wherein the cannula hub lock comprises two extensions extending in the direction transverse to the longitudinal axis, wherein the two extensions are configured to be spaced apart from one another, wherein the clip body is extending in a circumferential direction around the longitudinal axis from one of the two extensions to the other of the two extensions, wherein the inner section of the body is engaged with the two extensions when the inner section of the body is coupled to the clip, and wherein the inner section of the body is disengaged from the extensions when the inner section of the body is decoupled from the clip.

27. The cannula assembly of claim 17, wherein the outer section of the body of the lock holder is a user-accessible button such that the lock holder is moved by a user of the cannula assembly manually to causes the cannula hub lock to be moved from the locked position to the released position.

28. The cannula assembly of claim 17, wherein the outer section of the body of the lock holder is configured to be positioned within a gas-tight chamber such that the lock holder is moved by a change of the pneumatic pressure within the gas-tight chamber to cause the cannula hub lock to be moved from the locked position to the released position.

29. The cannula assembly of claim 16, the housing comprises a container fastener configured to be operably attached to the infusion container, wherein the container fastener is nearby the container end of the housing, and wherein the container fastener is at least one of a thread, a bayonet connector, and a snap-fit connector.

30. The cannula assembly of claim 16, comprising a penetrable cap membrane tightly connected to a tip of the cannula, forming a cap around the tip of the cannula, before a soft membrane sealing the container end of the housing is pierced.
